(19) United States
(12) Patent Application Publication
(10) Pub. No.: US 2014/0230114 A1
(43) Pub. Date: Aug. 21, 2014

(54) Sun Shade Apparatus

(71) Applicant: Clifford C. Wilson, JR., Skillman, NJ (US)

(72) Inventor: Clifford C. Wilson, JR., Skillman, NJ (US)

(21) Appl. No.: 13/769,554

(22) Filed: Feb. 18, 2013

(51) Int. Cl. A61F 9/04

(52) U.S. Cl. CPC A61F 9/045
USPC 2/13

(57) ABSTRACT

A sun shade apparatus for use with eyeglasses having a pair of temples and worn relative to a user's head includes a flexible headband removably coupled to the pair of temples of the eyeglasses. A head shade assembly is coupled to an upper end of the framework so as to be displaced from the headband and above the user's head, the head shade assembly having a generally planar configuration and made of a generally opaque material configured to block sun rays from the user's head. The head shade assembly includes a plurality of wire frame members that are selectively bendable to selectively orient the sun shade apparatus relative to the user's head. The sun shade apparatus may include a coupled to a lower surface of the head shade assembly that may be powered by solar or battery power.

### BACKGROUND OF THE INVENTION

This invention relates to devices that provide shade from the sun and, more particularly, to a sun shading apparatus that provides shade to a person's head from the sun's rays while remaining in proximity to but displaced from a user's head.

A person often desires to shade his face and head from the intense rays from the sun, especially in the summer season. For instance, watching a sporting event can become very uncomfortable when a user is subjected to intense sunlight for several hours. In addition to being uncomfortable, exposure of a person's head to intense and prolonged sunlight may result in an increase in body temperature, profuse sweating, dehydration, or even skin cancer.

Perhaps the most common device utilized to block the sun's rays is a baseball-style hat. This type of head covering typically covers the top, sides, and back of a wearer's head and includes a brim that extends forwardly so as to cast shade or a shadow on the wearer's face. Although assumably effective for the intended purpose of shielding the wearer's head from sunlight, a traditional hat captures heat between the wearer's head and the hat itself, leading to perspiration, a rise in temperature, and overall discomfort. In such circumstances, the user may need to remove the sun shielding hat periodically just to enable heat to dissipate. The user may, in fact, seek shade that does not contact his head at all, such as a canopy.

Therefore, it would be desirable to have a sun shield apparatus to be worn on the head of a user that shields the user's head from the sun while not actually contacting the user's head at all. Further, it would be desirable to have a sun shield apparatus that may be coupled to a user's eyeglasses or sunglasses and supported above and displaced from the user's head. In addition, it would be desirable to have a sun shield apparatus that is flexible and adjustable to shield a user's head from sunlight regardless of the sun's position. It would also be desirable to have a sun shield apparatus having a fun for blowing air either onto or away from a user's head.

### SUMMARY OF THE INVENTION

A sun shade apparatus according to the present invention for use with eyeglasses having a pair of temples and worn relative to a user's head includes a flexible headband removably coupled to the pair of temples of the eyeglasses. A head shade assembly is coupled to an upper end of said framework so as to be displaced from said headband and above the user's head, said head shade assembly having a generally planar configuration and made of a generally opaque material configured to block sun rays from the user's head. The head shade assembly includes a plurality of wire frame members that are selectively bendable to selectively orient said sun shade apparatus relative to the user's head. The sun shade apparatus may include a coupled to a lower surface of said head shade assembly that may be powered by solar or battery power.

Therefore, a general object of this invention is to provide a sun shade apparatus that provides shade from the sun's rays while remaining in proximity to but displaced from a user's head.

Another object of this invention is to provide a sun shade apparatus, as aforesaid, in which a head shade assembly is coupled to and supported by a user's eyeglasses or sunglasses.

Yet another object of this invention is to provide a sun shade apparatus, as aforesaid, in which the head shade assembly is adjustable to shield the sun's rays from different angles.

A further object of this invention is to provide a sun shade apparatus, as aforesaid, having a fan for blowing air onto or away from a user's head when wearing the sun shade apparatus.

A still further object of this invention is to provide a sun shade apparatus, as aforesaid, that includes a solar panel configured to collect solar energy for powering the fan.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a sun shade apparatus according to a preferred embodiment of the present invention;

FIG. 2 is a side view of the sun shade apparatus as in FIG. 1;

FIG. 3 is a top view of the sun shade apparatus as in FIG. 1;

FIG. 4 is a top view of the sun shade apparatus with a solar panel as in FIG. 1; and

FIG. 5 is a side view of the sun shade apparatus with a fan as in FIG. 1.

### DESCRIPTION OF THE PREFERRED EMBODIMENT

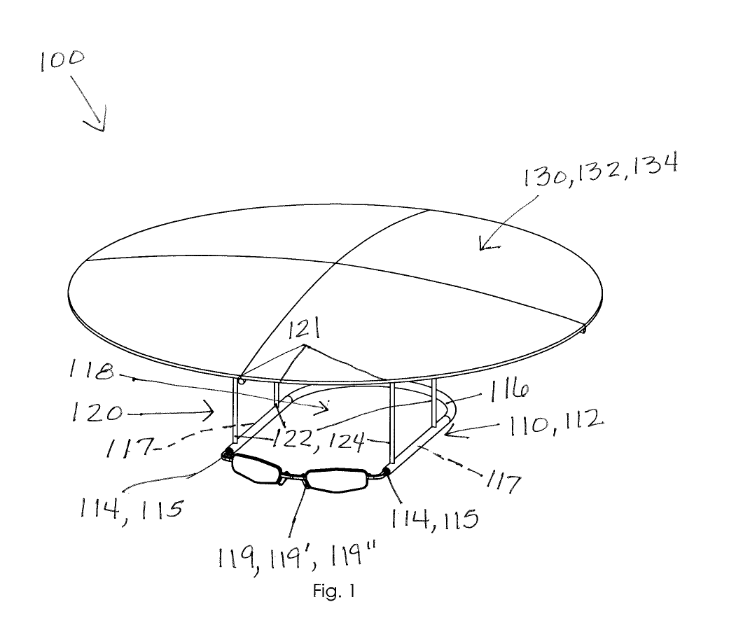

Sun shade apparatuses according to the present invention will now be described in detail with reference to FIGS. 1 through 5 of the accompanying drawings. More particularly, a sun shade apparatus 100 according to one embodiment includes a headband 110, a framework 120 and a head shade assembly 130.

As shown in FIG. 1, the headband 110 includes a generally tubular configuration 112 having opposed ends 114 and a body portion 116 extending between the opposed ends 114. The opposed ends 114 are open ends 115 and the body portion 116 defines an open area 118, the open ends 115 being configured to selectively receive a pair of temples 117 of eyeglasses 119 into the body portion 116. The body portion 116 is configured to extend about a back portion of a user's head when received into the body portion 116 though not explicitly shown in the drawings. The headband 110 may include a flexible construction coupled to the pair of temples 117 of the eyeglasses 119. The eyeglasses 119 shown in FIG. 1 may be corrective eyewear 119′, sunglasses 119″ or any type of eyeglasses. The headband 110 may be made of any flexible material, such as polychloroprene or Neoprene®. The framework 120 is coupled to the headband 110 and extends upwardly therefrom.

The framework 120 includes four (4) stems 122, although the framework 120 may include any number of stems. The stems 122 more specifically may be chenille stems 124 or any type of stem. The framework 120 may also be any length and thickness to shield a user from the sun or any other light source.

The head shade assembly 130 is operatively coupled to an upper end 121 of the framework 120 so as to be displaced from the headband 110 and above a user's head, though not explicitly shown in the drawings. The head shade assembly 130 has a generally planar configuration 132 and may be made of a generally opaque material 134 so as to block sun rays from the user's head. The opaque material may be generally inflexible such in a manner as the brim of a ball cap. Alternatively, the generally opaque material 134 may be made of a flexible, lightweight and cloth-covered material, such as 1,5,7-triazabicyclo [4.4.0] dec-5-ene or the like.

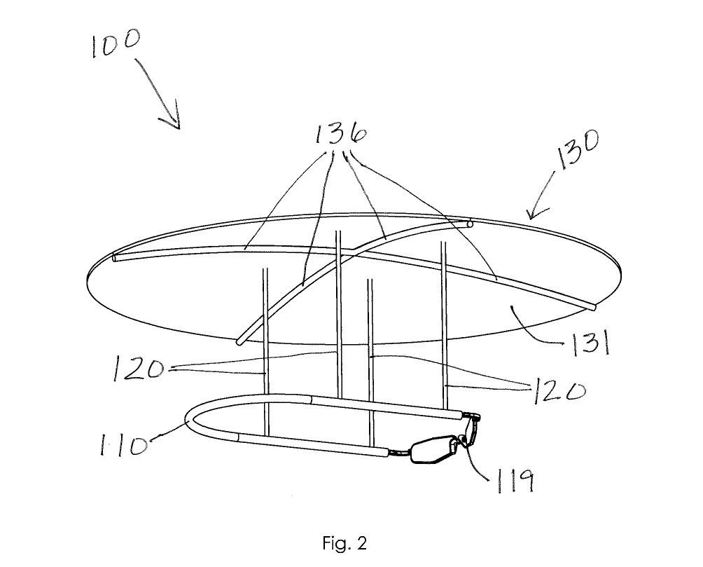

As shown in FIG. 2, the head shade assembly 130 may include a plurality of wire frame members 136 that are bent around the user's head to removably secure the sun shade apparatus 100 to the user's head, though not explicitly shown in the drawings. FIG. 2 shows the wire frame members 136 on a lower surface 131 of the head shade assembly 130.

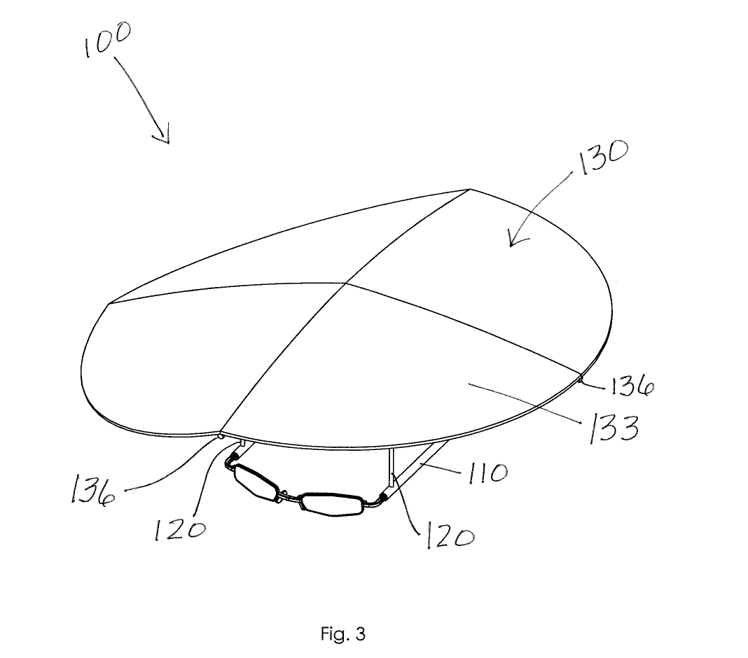

As shown in FIG. 3, the head shade assembly 130 includes wire frame members 136 that are selectively bendable to selectively orient the sun shade apparatus relative to the user's head, though not explicitly shown in the drawings. FIG. 2 shows the wire frame members 136 on a lower surface 131 of the head shade assembly 130.

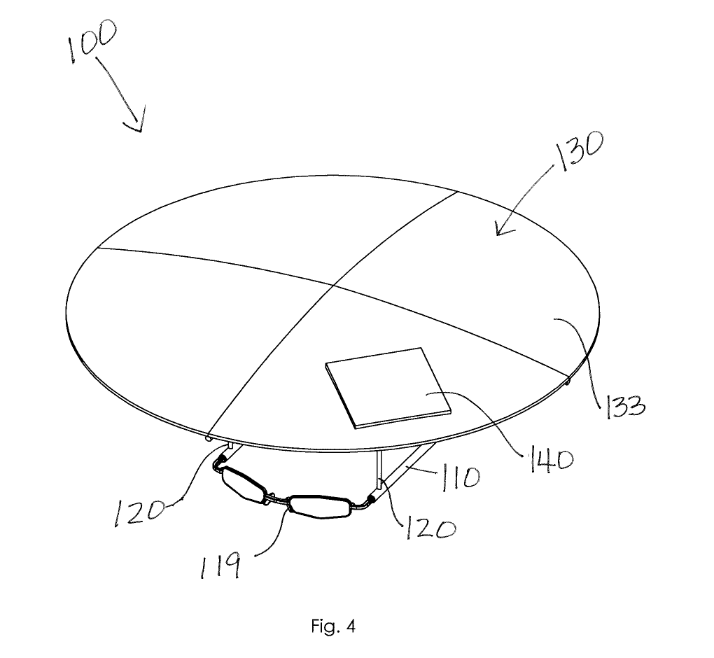

As shown in FIG. 4, a solar panel 140 may be operatively coupled to the upper surface 133 of the head shade assembly 130. The solar panel 140 receives sunlight and converts light energy from sunlight into electrical energy. The solar panel 140 may be electrically connected to the fan 150 as described below.

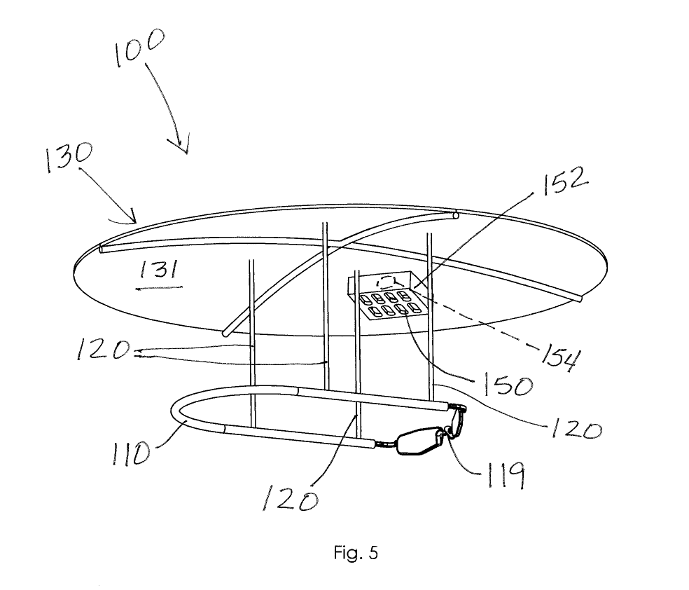

As shown in FIG. 5, a fan 150 may be operatively coupled to the lower surface 131 of the head shade assembly 130. The fan 150 is typically an electric powered fan 152 but may be any type of fan. The fan 150 may be configured to provide forced air onto a user's head or to direct warm air away from a user's head to assist cooling the user. The solar panel 140 may be in electrical communication with the fan 150 with one or more electrical wires 152 and may provide electrical energy to the fan 150 when the solar panel 140 is exposed to sunlight to thereby generate electrical energy to be transmitted to the fan 150. The fan 150 may also be powered with one or more batteries 154 of any type such as alkaline batteries, lithium batteries or polymer ion batteries and any size.

In use, the sun shade apparatus 100 can shield a person's head from sunlight without the shade assembly actually touching a user's head. In other words, the sun shade apparatus 100 allows heat to escape from the user's head and is not like a hat that traps heat against a user's head. The sun shade apparatus 100 is suspended from the eyeglasses 119 via four chenille stems 124 disposed two per side. These chenille stems 124 may be permanently attached to a thin NEOPRENE® headband 110 which may fit over or receive the temples 117 of the eyeglasses 119 and extend around the back of the user's head. This would provide the dual benefit of holding the suspended head shade assembly 130 in place, as well as preventing the eyeglasses 119 from being dislodged by wind or other mishap. An alternative embodiment would be to attach the stems (not shown) to the temples 117 of the eyeglasses 119 with small rubber or plastic loops (not shown). This method would be less costly to manufacture and would leave less material in contact with the user's head. However, the resulting set-up would be less stable and could lead to problems with wind. Another embodiment includes a small, lightweight fan 150 coupled to a lower surface 131 of the head shade assembly 130 to assist in circulating air. The fan 150 may be solar-powered by a solar panel 140 coupled to an upper surface 133 of the head shade assembly 130 or powered by one or more batteries 154.

It is understood that while certain forms of this invention have been illustrated and described, it is not limited thereto except insofar as such limitations are included in the following claims and allowable functional equivalents thereof.

1. A sun shade apparatus to be used with eyeglasses having a pair of temples and worn relative to a user's head, comprising:
a framework operatively coupled to the pair of temples of the eyeglasses; and
a head shade assembly coupled to an upper end of said framework so as to be upwardly displaced from the temples of the eyeglasses and above the user's head, said head shade assembly having a generally planar configuration and constructed of a generally opaque material so as to block sun rays from the user's head.

2. The sun shade apparatus as in claim 1, further comprising a flexible headband removably coupled to the pair of temples of the eyeglasses, said headband including a generally tubular configuration having opposed ends and a body portion extending between said opposed ends;
wherein:
said opposed ends of said headband are open and said body portion defines an open area, said open ends being configured to selectively receive the pair of temples of the eyeglasses into said body portion, said body portion being configured to extend about a back portion of the user's head when received into said body portion; and
said framework is coupled to said headband and extends upwardly therefrom so as to displace said head shade assembly above said headband.

3. The sun shade apparatus as in claim 2, wherein said headband is constructed of polychloroprene.

4. The sun shade apparatus as in claim 1, wherein said head shade assembly includes a plurality of wire frame members that are selectively bendable to selectively orient said sun shade apparatus relative to the user's head.

5. The sun shade apparatus as in claim 1, wherein said framework includes four stems.

6. The sun shade apparatus as in claim 5, wherein said four stems include four chenille stems.

7. The sun shade apparatus as in claim 1, wherein said generally opaque material is generally inflexible.

8. The sun shade apparatus as in claim 1, wherein said generally opaque material is generally flexible, lightweight, and cloth-covered, said opaque material including 1,5,7-Triazabicyclo [4.4.0] dec-5-ene.

9. The sun shade apparatus as in claim 1, wherein said eyeglasses are selected from a group including corrective eyewear and sunglasses.

10. A sun shade apparatus for use with eyeglasses having a pair of temples and worn relative to a user's head, comprising:
a flexible headband removably coupled to the pair of temples of the eyeglasses;
a framework coupled to said headband and extending upwardly therefrom;
a head shade assembly coupled to an upper end of said framework so as to be displaced from said headband and above the user's head, said head shade assembly having a generally planar configuration and made of a generally opaque material configured to block sun rays from the user's head and said head shade assembly includes a plurality of wire frame members that are selectively bendable to selectively orient said sun shade apparatus relative to the user's head; and
a fan coupled to a lower surface of said head shade assembly.

11. The sun shade apparatus as in claim 10, wherein said headband includes a generally tubular configuration having opposed ends and a body portion extending between said opposed ends, said opposed ends being open and said body portion defining an open area, said open ends being configured to selectively receive the pair of temples of the eyeglasses into said body portion, said body portion configured to extend about a back portion of the user's head when received into said body portion.

12. The sun shade apparatus as in claim 10, wherein said headband is made of polychloroprene.

13. The sun shade apparatus as in claim 10, wherein said framework includes four stems.

14. The sun shade apparatus as in claim 13, wherein said four stems include four chenille stems.

15. The sun shade apparatus as in claim 10, wherein said generally opaque material is flexible, lightweight and cloth-covered.

16. The sun shade apparatus as in claim 15, wherein said generally opaque material is made of 1,5,7-Triazabicyclo [4.4.0] dec-5-ene.

17. The sun shade apparatus as in claim 10, further comprising a solar panel coupled to an upper surface of said head shade assembly and configured to receive sunlight and convert said sunlight into electrical energy.

18. The sun shade apparatus as in claim 17, wherein said solar panel is electrically connected to said fan and configured to provide said electrical energy thereto.

19. The sun shade apparatus as in claim 10, wherein said fan is powered by one or more batteries.

20. The sun shade apparatus as in claim 10, wherein said eyeglasses are selected from a group including corrective eyewear and sunglasses.
